(19) United States
(12) Patent Application Publication
(10) Pub. No.: US 2021/0120974 A1
(43) Pub. Date: Apr. 29, 2021

(54) SAFETY APPARATUS FOR INFANT BED

(71) Applicant: YEHIEL FREISLER, EILAT (IL)

(72) Inventor: YEHIEL FREISLER, EILAT (IL)

(21) Appl. No.: 16/737,951

(22) Filed: Jan. 9, 2020

(30) Foreign Application Priority Data
Oct. 23, 2019 (IL) 270129

(51) Int. Cl. A47D 15/00

(52) U.S. Cl. CPC A47D 15/00

(57) ABSTRACT

A safety apparatus for an infant bed, including a first sensor, for sensing a distance of an infant disposed within the bed from a door thereof, a second sensor, for sensing a distance of an adult disposed outside the bed from the door, a third sensor, for sensing that the door is open, and an alarming gadget, for alarming in case, as sensed by the sensors, the door is open, and the adult's distance is sufficiently large, and the infant's distance is sufficiently small.

### CROSS-REFERENCE TO RELATED APPLICATIONS

This application claims the benefit of priority from Israel Application No. 270129, filed Oct. 23, 2019, the disclosure of which is incorporated herein by reference.

### TECHNICAL FIELD

The invention relates to the field of infant beds.

### BACKGROUND

There is a long felt need for apparatus that an infant does not fall out of the infant bed.

### SUMMARY

A safety apparatus for an infant bed, including:

an alarming gadget (54), for alarming in case, as sensed by sensors that, a door is open, and the adult's distance is sufficiently large, and the infant's distance is sufficiently small.

### BRIEF DESCRIPTION OF THE DRAWINGS

Embodiments, features, and aspects of the invention are described herein in conjunction with the following drawings:

FIG. 1 is a top view of an infant bed including a safety apparatus according to one embodiment of the invention.

FIG. 2 is the top view of the infant bed including the safety apparatus of FIG. 1 in another case.

FIG. 3 is the top view of the infant bed including the safety apparatus of FIG. 1 in another case.

FIG. 4 is a perspective view of the infant bed including the safety apparatus of FIG. 1.

FIG. 5 magnifies the safety apparatus of FIG. 4.

FIG. 6 is the top view of the infant bed including the safety apparatus of FIG. 1 in another case.

FIG. 7 is the top view of the infant bed including the safety apparatus of FIG. 1 in another case.

FIG. 8 is the top view of the infant bed including the safety apparatus of FIG. 1 in another case.

The drawings are not necessarily drawn to scale.

### DETAILED DESCRIPTION

The invention will be understood from the following detailed description of embodiments of the invention, which are meant to be descriptive and not limiting. For the sake of brevity, some well-known features are not described in detail.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

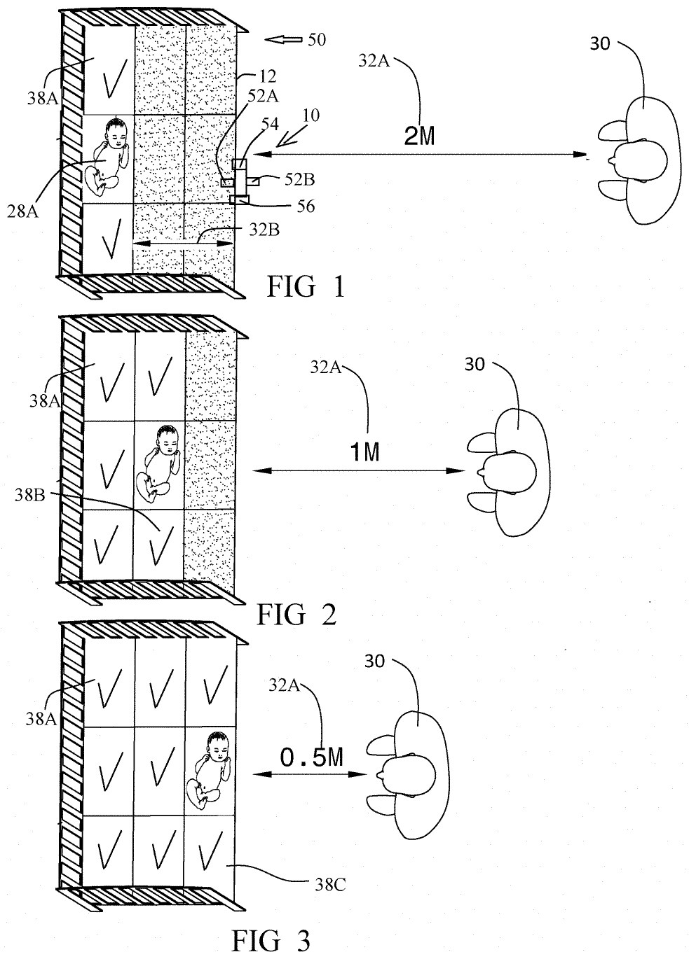

FIG. 1 is a top view of an infant bed including a safety apparatus according to one embodiment of the invention.

A safety apparatus 10 for an infant bed 50 includes a sensor 52A, for sensing the infant location; a sensor 52B, for sensing the adult location; a sensor 56, for sensing that the door 12 is open; and an alarming gadget 54.

According to one example, in case door 12 is open, and adult 30 is located 2 meters away, the infant may be located only in the inner zone 38A, being far from the open door. Otherwise, alarming gadget 54 alarms.

FIG. 2 is the top view of the infant bed including the safety apparatus of FIG. 1 in another case.

And in case door 12 is open, and adult 30 is located 1 meter away, the infant may be located in inner zone 38A, and as well in the central zone 38B.

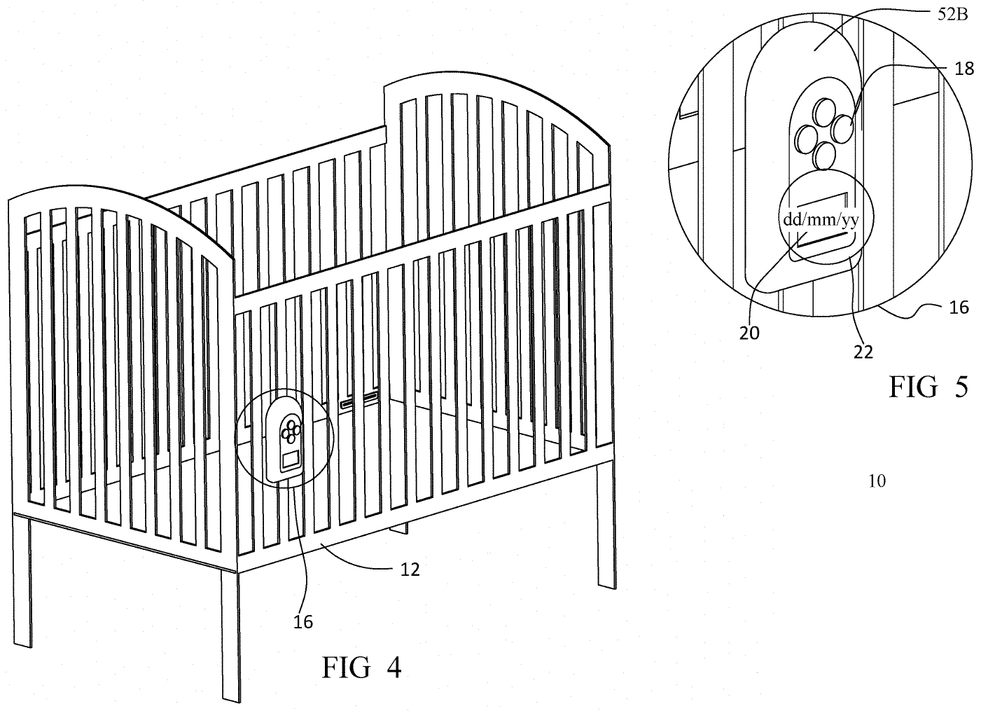

FIG. 3 is the top view of the infant bed including the safety apparatus of FIG. 1 in another case.

In case door 12 is open, and adult 30 is located 0.5 meter away, the infant may be located in any zone.

The above examples may apply to a 2 month old infant. However, the parameters are a function of the age.

FIG. 4 is a perspective view of the infant bed including the safety apparatus of FIG. 1.

FIG. 5 magnifies the safety apparatus of FIG. 4.

Safety apparatus 10 includes a user interface 18 for inputting the infant's birth date 20.

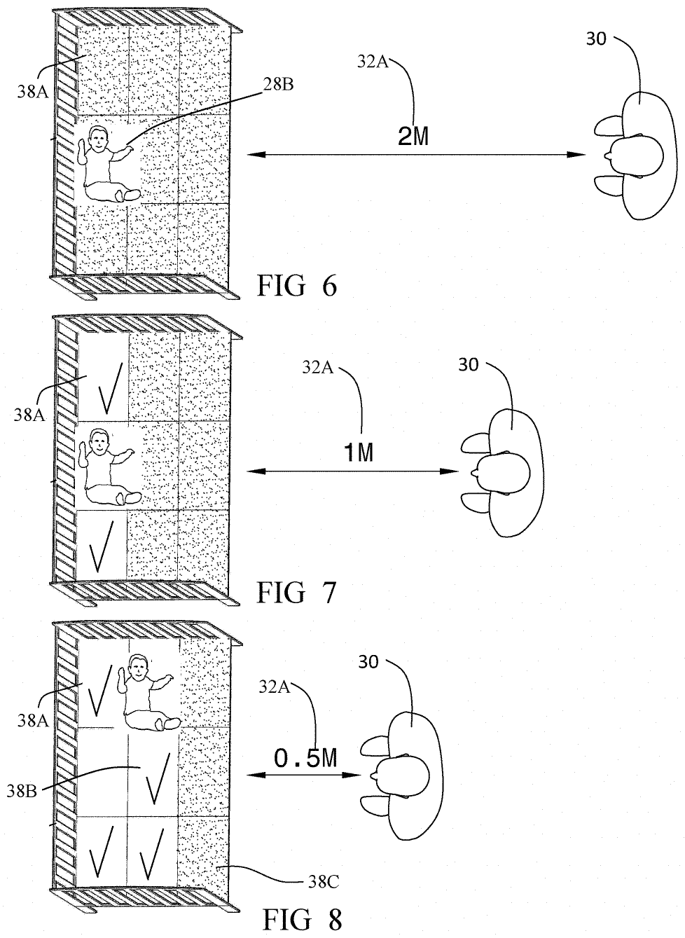

FIG. 6 is the top view of the infant bed including the safety apparatus of FIG. 1 in another case.

In case door 12 is open, and the infant has grown, such as to sit, and adult 30 is located 2 meters away, the alarm is turned on wherever the infant is located.

FIG. 7 is the top view of the infant bed including the safety apparatus of FIG. 1 in another case.

In case door 12 is open, and the infant has grown, such as to sit, and adult 30 is located 1 meter away, the infant may be located only in the inner zone 38A; otherwise, alarming gadget 54 alarms.

FIG. 8 is the top view of the infant bed including the safety apparatus of FIG. 1 in another case.

In case door 12 is open, and adult 30 is located 0.5 meter away, the infant may be located in inner zone 38A, and as well in the central zone 38B.

Thus, in one aspect, the invention is directed to a safety apparatus (10) for an infant bed (50), including:
a first sensor (52A), for sensing a distance (32A) of an infant (28A) disposed within the bed (50) from a door (12) thereof;
a second sensor (52B), for sensing a distance (32B) of an adult (30) disposed outside the bed (50) from the door (12);
a third sensor (56), for sensing that the door (12) is open; and
an alarming gadget (54), for alarming in case, as sensed by the sensors (52A,52B,56), the door (12) is open, and the adult's distance (32A) is sufficiently large, and the infant's distance is sufficiently small.

The sufficiently large adult's distance (32A) and the sufficiently small infant's distance (32B) include a function of one another.

The safety apparatus (10) may further include:
a user interface (18), for obtaining an age of the infant,
   wherein the sufficiently large adult's distance (32A) and the sufficiently small infant's distance (32B) include functions of the infant's age.

In the figures and/or description herein, the following reference numerals (Reference Signs List) have been mentioned:
numeral 10 denotes the safety apparatus for infant bed 50 according to one embodiment of the invention;
12: door;
18: user interface;
20: inputted birth date for obtaining age;
28A,28B: infant;
32A,32B: distances;
38A,38B,38C: zones within bed 50;
50: infant bed;
52A,52B: distance sensors;
54: alarming gadget;

The foregoing description and illustrations of the embodiments of the invention have been presented for the purpose of illustration, and are not intended to be exhaustive or to limit the invention to the above description in any form.

Any term that has been defined above and used in the claims, should be interpreted according to this definition.

The reference numbers in the claims are not a part of the claims, but rather used for facilitating the reading thereof. These reference numbers should not be interpreted as limiting the claims in any form.

What is claimed is:

1. A safety apparatus for an infant bed, comprising:
a first sensor, for sensing a distance of an infant disposed within said bed from a door thereof;
a second sensor, for sensing a distance of an adult disposed outside said bed from said door;
a third sensor, for sensing that said door is open; and
an alarming gadget, for alarming in case, as sensed by said sensors, said door is open, and said adult's distance is sufficiently large, and said infant's distance is sufficiently small.

2. The safety apparatus according to claim 1, wherein said sufficiently large adult's distance and said sufficiently small infant's distance comprise a function of one another.

3. The safety apparatus according to claim 1, further comprising:
a user interface, for obtaining an age of said infant,
wherein said sufficiently large adult's distance and said sufficiently small infant's distance comprise functions of said infant's age.
